(19) United States
(12) Patent Application Publication
(10) Pub. No.: US 2012/0045269 A1
(43) Pub. Date: Feb. 23, 2012

(54) FLUID TUBE

(76) Inventor: YING-SHU LIU, JIANGSU (CN)

(21) Appl. No.: 13/187,571

(22) Filed: Jul. 21, 2011

(30) Foreign Application Priority Data
Aug. 23, 2010 (CN) 201020299840.X

(51) Int. Cl. A61F 13/40

(52) U.S. Cl. 401/134

(57) ABSTRACT

A fluid tube includes a tube, a fluid, for example, a fluid medicine, alcohol or cosmetic fluid carried in the tube, a sealing cap heat-sealed to one end of the tube and having dentations breakable for letting the atmospheric pressure enter the tube, and a plug cap capped on the other end of the tube to seal the fluid in said tube and having rounded inside protrusions forced into friction engagement with the tube.

### BACKGROUND OF THE INVENTION

1. Field of the Invention

The present invention relates to fluid containers and more particularly, to a fluid tube for carrying a fluid and keeping it well sealed for easy application.

2. Description of the Related Art

Many fluid containers with an attached applicator stick have been disclosed and are commercially available for electronic industrial cleaning application, cosmetic application and management or skin treatment application. The applicator stick of a fluid container is attached to the cap of the container and dipped in the contained fluid. During application, the fluid may drop from the stick applicator accidentally, causing contamination.

There is known a fluid container comprising a tubular container body having a close end and an open end, and a seal sealing the open end. The tubular container body has a breakable neck. When in use, the user can break the breakable neck, enabling the fluid to flow out of the tubular container body for application. This design of fluid container is convenient for delivery and carrying. However, when breaking the breakable neck of the tubular container body, the fluid may be splashed onto the surroundings, causing contamination.

China Patent No. 2009201879186 discloses a fluid tube having two different fluids contained in a tube. One fluid works as a floating plug. The fluid working as a floating plug is suspending the bottom open end of the tube and kept in close contact with the other fluid in the tube. The top open end of the tube is sealed by a sealing cap that has inside protrusions forced into friction engagement with the tube. Under the normal condition, the sealing cap is capped on one end of the tube. By means of friction engagement between the inside protrusions of the seal cap and the wall of the tube, the respective end of the tube is well sealed by the sealing cap, protecting the contained fluid against floating or evaporation. When pull the sealing cap away from the tube, the atmosphere pressure immediately enters the tube, allowing the floating plug to float and to force the other fluid out of the tube for application. This design of fluid tube is convenient for delivery, carrying and application. Further, the sealing cap can be repeatedly used. However, the friction engagement between the sealing cap and the tube achieves an interference fit. A user must employ much effort to open the sealing cap from the tube. Further, during a use, the fluid of the floating plug may contaminate the surface to be cleaned.

Therefore, it is desirable to provide a fluid tube that eliminates the aforesaid problems.

### SUMMARY OF THE INVENTION

The present invention has been accomplished under the circumstances in view. It is one object of the present invention to provide a fluid tube carrying a fluid, which well seals the contained fluid against floating or evaporation, facilitating quick application.

To achieve this and other objects of the present invention, a fluid tube comprises a tube having two opposing open ends, a fluid contained in the tube, a sealing cap heat-sealed to one end of the tube, and a plug cap capped on the other end of the tube to seal the fluid in the tube and to protect the fluid against evaporation.

Further, the sealing cap comprises a plurality of dentations breakable by an external pressure. Further, the plug cap comprises a plurality of rounded inside protrusions configured for friction engagement with the inside wall or outside wall of the tube. Thus, the fluid tube is convenient for delivery or carrying by the user. During application, impart a pressure to the sealing cap to break the dentations, tear off the sealing cap, or deplug the plug cap from the tube, letting the atmospheric pressure enter the tube to force the contained fluid downwardly along the tube toward the outside for application. Further, the plug cap is reusable without causing contamination. The installation of the fluid tube is easy and simple.

### BRIEF DESCRIPTION OF THE DRAWINGS

FIG. 1 is a sectional side view of a fluid tube in accordance with a first embodiment of the present invention.

FIG. 2 is a bottom view in an enlarged scale of the fluid tube in accordance with the first embodiment of the present invention.

FIG. 3 is a sectional side view of a fluid tube in accordance with a second embodiment of the present invention.

FIG. 4 is an enlarged view of a part of the second embodiment of the present invention, illustrating the rounded inside protrusions kept in friction engagement with the outside wall of the tube.

FIG. 5 is a sectional side view of a fluid tube in accordance with a third embodiment of the present invention.

FIG. 6 is an enlarged view of a part of the third embodiment of the present invention, illustrating the rounded inside protrusions kept in friction engagement with the inside wall of the tube.

### DETAILED DESCRIPTION OF THE PREFERRED EMBODIMENT

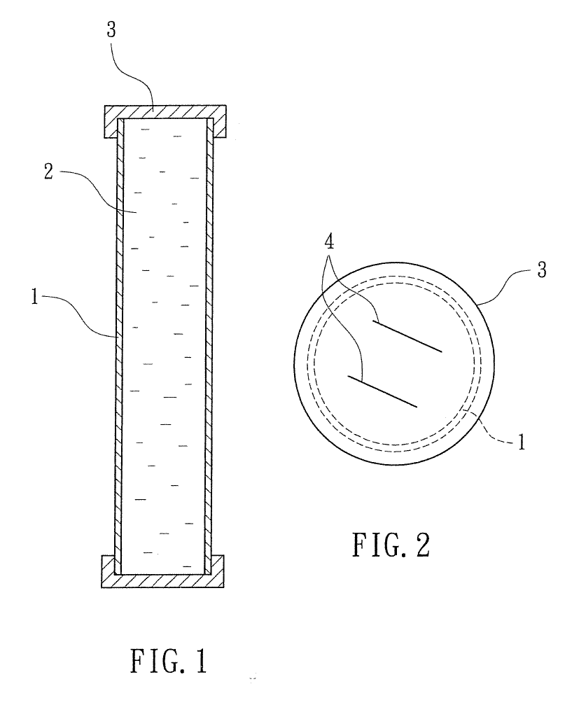

Referring to FIGS. 1 and 2, a fluid tube in accordance with a first embodiment of the present invention is shown comprising a tube 1, a fluid 2 that can be, for example, a fluid medicine, alcohol, a cosmetic fluid carried in the tube 1, and a sealing cap 3 capped on one open end (or each of two opposite open ends) of the tube 1 to protect the fluid 2 against the effect of the outside atmospheric pressure. The sealing cap 3 has dentations 4.

When using the fluid tube, impart a pressure to the sealing cap 3 to break the dentations 4 with, for example, the thumb, letting the atmospheric pressure enter the tube 1 to force the contained fluid 2 downwardly along the tube 1 toward the outside for application when the tube 1 is kept in vertical or inclined.

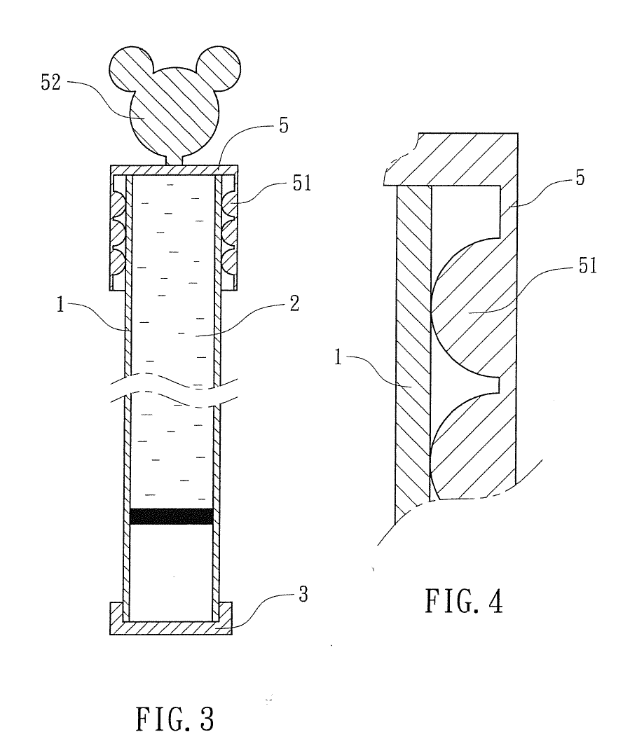

Referring to FIGS. 3 and 4, a fluid tube in accordance with a second embodiment of the present invention is shown comprising a tube 1, a fluid 2 carried in the tube 1, a sealing cap 3 heat-sealed to one open end of the tube 1 to protect the fluid 2 against the effect of the outside atmospheric pressure, and a plug cap 5 capped on the other open end of the tube 1. The plug cap 5 has a finger lug 52 located on the outside, and rounded inside protrusions 51 located on the inside for friction engagement with the outside wall of the corresponding open end of the tube 1. Similar to the aforesaid first embodiment, the sealing cap 3 in accordance with this second embodiment has dentations (not shown) on it. When using the fluid tube, impart a pressure to the sealing cap 3 to break the dentations, letting the atmospheric pressure enter one end of the tube 1 to force the contained fluid 2 downwardly along the tube 1 toward the outside for application.

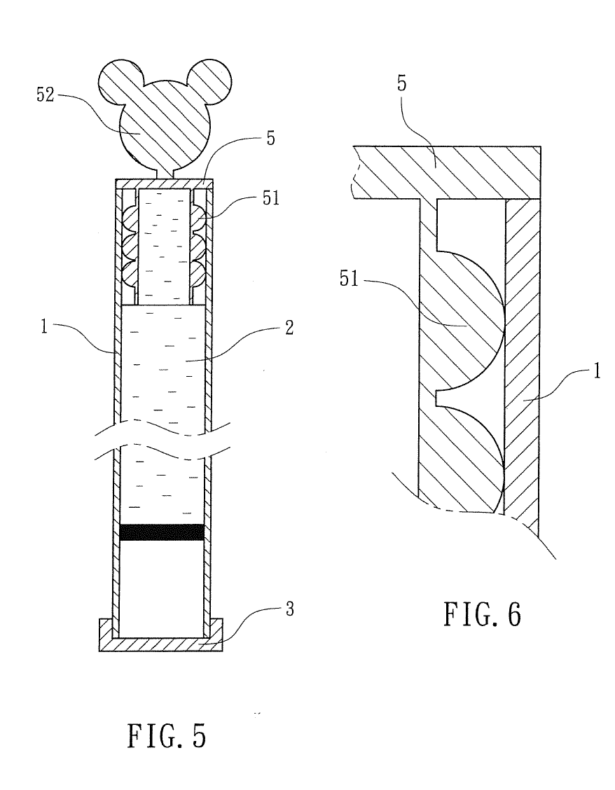

Referring to FIGS. 5 and 6, a fluid tube in accordance with a third embodiment of the present invention is shown comprising a tube 1, a fluid 2 carried in the tube 1, a sealing cap 3 heat-sealed to one open end of the tube 1 to protect the fluid 2 against the effect of the outside atmospheric pressure, and a plug cap 5 capped on the other open end of the tube 1. The plug cap 5 according to this third embodiment is a male plug member press-fitted into the corresponding open end of the tube to force the rounded inside protrusions 51 thereof into friction engagement with the inside wall of the tube 1. Similar to the aforesaid first and second embodiments, the sealing cap 3 in accordance with this third embodiment has dentations (not shown) on it. When using the fluid tube, impart a pressure to the sealing cap 3 to break the dentations, letting the atmospheric pressure enter one end of the tube 1 to force the contained fluid 2 downwardly along the tube 1 toward the outside for application.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A fluid tube, comprising:
a tube having two opposing open ends;
a fluid contained in said tube;
a sealing cap heat-sealed to one open end of said tube; and
a plug cap capped on the other open end of said tube to seal said fluid in said tube, said plug cap comprising a plurality of rounded inside protrusions.

2. The fluid tube as claimed in claim 1, wherein said sealing cap comprises a plurality of dentations breakable by an external pressure.

3. The fluid tube as claimed in claim 1, wherein said rounded inside protrusions of said plug cap are configured for friction engagement with said tube.

4. The fluid tube as claimed in claim 1, wherein said rounded inside protrusions of said plug cap are configured for friction engagement with an inside wall of said tube.

5. The fluid tube as claimed in claim 1, wherein said rounded inside protrusions of said plug cap are configured for friction engagement with an outside wall of said tube.
